(19) United States
(12) Patent Application Publication
(10) Pub. No.: US 2025/0025511 A1
(43) Pub. Date: Jan. 23, 2025

(54) TREATMENT OF INCLUSION BODY MYOSITIS WITH ADIPOSE-DERIVED REGENERATIVE CELLS

(71) Applicant: University of Kansas, Lawrence, KS (US)

(72) Inventors: Richard J. Barohn, Columbia, MO (US); Mazen M. Dimachkie, Overland Park, KS (US); Rupal Soder, Overland Park, KS (US)

(21) Appl. No.: 18/779,564

(22) Filed: Jul. 22, 2024

Related U.S. Application Data
(60) Provisional application No. 63/514,980, filed on Jul. 21, 2023.

(51) Int. Cl. A61K 35/28; C12N 5/0775; G01N 33/68

(52) U.S. Cl. CPC A61K 35/28; C12N 5/0667; G01N 33/6887; G01N 2800/52

(57) ABSTRACT

A method for treating inclusion body myositis (IBM) in a subject can include: providing a subject that has IBM; obtaining adipose-derived regenerative cells (ADRC); and administering the ADRC to the subject to treat the IBM. The ADRCs can be obtained by a closed system for use as the therapeutic as described herein. The ADRCs can be obtained in a system that can then be used for providing the ADRCs for administration. The system can be automated and may provide the ADRCs in sufficient purity for reimplantation. The method can include: injecting the ADRC as cells into one injection location or a plurality of injection locations to provide at least a million cells, more preferably at least 10 million cells, even more preferably at least 20 million cells, or most preferably at least 30 million cells.

### CROSS-REFERENCE TO RELATED APPLICATIONS

This patent application claims priority to U.S. Provisional Application No. 63/514,980 filed Jul. 21, 2023, which provisional is incorporated herein by specific reference in its entirety.

### BACKGROUND

### Field

The present disclosure relates to adipose-derived regenerative cells (ADRC) for use as a treatment for inclusion body myositis (IBM).

### Description of Related Art

Inclusion body myositis (IBM) is an inflammatory muscle disease that occurs in older adults. The IBM disease presents with slow progressive weakness and wasting of both proximal muscles (e.g., located on or close to the torso) and distal muscles (e.g., close to hands or feet). Sometimes IBM is first apparent in the finger flexors and knee extensors. In IBM, two processes appear to occur in the muscles in parallel, one autoimmune and the other degenerative. In some instances, inflammation is evident from the invasion of muscle fibers by immune cells. Also, degeneration is characterized by the appearance of holes, deposits of abnormal proteins, and filamentous inclusions in the muscle fibers. However, there is currently no approved treatment for IBM (Nogalska A et al., Sodium phenylbutyrate reverses lysosomal dysfunction and decreases amyloid-β42 in an in vitro-model of inclusion-body myositis, Neurobiology of Disease, Volume 65, 2014, Pages 93-101, ISSN 0969-9961, doi.org/10.1016/j.nbd.2014.01.012).

Pathological characteristics of IBM include vacuolated muscle fibers having: accumulations of multi-protein aggregates, including amyloid-β (Aβ) 42 and its toxic oligomers; increased γ-secretase activity; and impaired autophagy. Cultured human muscle fibers with experimentally-impaired autophagy recapitulate some of the IBM muscle abnormalities, including vacuolization and decreased activity of lysosomal enzymes, accompanied by increased Aβ42, Aβ42 oligomers, and increased γ-secretase activity. Sodium phenylbutyrate (NaPBA) is an orally bioavailable small molecule approved by the FDA for treatment of urea-cycle disorders. NaPB treatment reverses lysosomal dysfunction in an in vitro model of inclusion-body myositis, involving cultured human muscle fibers. NaPBA treatment improved lysosomal activity, decreased Aβ42 and its oligomers, decreased γ-secretase activity, and virtually prevented muscle-fiber vacuolization.

U.S. Publication No. 2022/0218818 teaches methods and compositions for treating an inflammatory disease or infectious disease in a subject in need thereof by administering to the subject a poxvirus and a stem cell, wherein the disease is not a cancer. The disease may be, for example, a chronic inflammatory disease (e.g., inclusion body myositis). The treatment method is directed to the administration of a poxvirus and a stem-cell (including adipose-derived regenerative cells) to the patient.

### SUMMARY

In some embodiments, a method for treating inclusion body myositis (IBM) in a subject can include: providing a subject that has IBM; obtaining adipose-derived regenerative cells (ADRC); and administering the ADRC to the subject to treat the IBM. The ADRCs can be obtained by a closed system for use as the therapeutic as described herein. The ADRCs can be obtained in a system that can then be used for providing the ADRCs for administration. The system can be automated and may provide the ADRCs in sufficient purity for reimplantation.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates a schematic diagram of an embodiment of a cell processing system.

FIG. 2 illustrates a flow diagram of an embodiment of a method of treating a patient with IBM using ADRCs.

FIG. 3 illustrates a flow diagram of an embodiment of a method of obtaining ADRCs.

FIG. 4 illustrates a flow diagram of an embodiment of a method of obtaining ADRCs.

FIG. 5 illustrates a flow diagram of an embodiment of a method of monitoring a biomarker.

FIG. 6 includes a graph of data for the monitoring of a biomarker.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology includes an autologous graft of adipose-derived regenerative cells (ADRCs) for the use in a therapy for inclusion body myositis (IBM). Currently, ADRCs are being investigated for treatment of IBM in an IBM-ADRC clinical study. The IBM-ADRC clinical study evaluates the safety and efficacy of the Celution® system in the processing of an autologous graft of adipose-derived regenerative cells (ADRCs) in the treatment of inclusion body myositis. The ADRCs are harvested, isolated, and reimplanted into a muscle or joint or proximal location so that the cells can have an impact on the areas affected by IBM. The muscle can be any muscle of the subject of a flange, hand, foot, limb, forelimb, hindlimb, neck or other muscle impacted by IBM, or joint space associated with such muscle.

### IBM

In some embodiments, the ADRCs can be administered to a subject having IBM. Accordingly, the subject has been diagnosed to have IBM, which can be characterized by progressive muscle disorder with inflammation, weakness, and atrophy of the muscles. IBM can also involve an abnormal immune response where the body's immune system attacks its own muscle tissue. Also, abnormal clumps of proteins (inclusion bodies) can accumulate in the muscle cells, which may play a role in muscle damage. It is thought that exposure to certain viruses or other environmental triggers might contribute to the onset of IBM.

In some embodiments, the symptoms used to diagnose a subject as having IBM can include muscle weakness, muscle atrophy, dysphagia, and impaired mobility. Progressive weakness occurs particularly in the muscles of the thighs, forearms, and fingers. IBM can cause difficulty with physical tasks, such as climbing stairs, getting up from a seated position, or gripping objects. There can be a noticeable shrinking of the affected muscles. The dysphagia can include difficulty swallowing, which can lead to choking or aspiration pneumonia. Often, IBM subjects have impaired mobility with increased risk of falls due to muscle weakness and atrophy. There can also be a loss of fine motor skills with difficulty with precise movements, such as buttoning a shirt or turning a key. Some individuals may experience muscle cramps and pain, although this is less common. IBM can cause a general feeling of tiredness or fatigue, often due to muscle weakness and the effort required to perform everyday activities. In some aspects of the invention, the treatment described herein can inhibit, stop, or improve these symptoms.

In some embodiments, the subject can be diagnosed to have IBM prior to the treatments described herein. In some aspects, a biopsy can reveal characteristic inclusion bodies and inflammation in muscle tissue. Additionally, electromyography (EMG) can be used to measure the electrical activity of muscles and can help differentiate IBM from other muscle disorders. Blood tests can be used to detect elevated levels of certain muscle enzymes, such as creatine kinase (CK), can indicate muscle damage. Also, an MRI can be used to show patterns of muscle inflammation and atrophy.

In some embodiments, the treatment described herein with ADRCs can be combined with other treatments. Such other treatments can include: physical therapy to maintain mobility and strength; occupational therapy to assist with daily activities; speech therapy for those with swallowing difficulties; administering immunosuppressive medications, and use of assistive devices, such as canes or wheelchairs, to aid mobility.

### ADRC

The ADRCs are a type of stem cell obtained from adipose (e.g., fat) tissue that have the potential to differentiate into various cell types, which makes them valuable for regenerative medicine. Often, ADRCs can be isolated from adipose tissue, which is abundant and relatively easy to obtain through procedures like liposuction. Typically, the adipose includes a heterogeneous mix of cells, such as adipose-derived stem cells (ADSCs), endothelial cells, smooth muscle cells, and immune cells, where the ADSCs can include ADRCs. ADRCs are multipotent in that they can differentiate into multiple cell types, including adipocytes (fat cells), osteocytes (bone cells), chondrocytes (cartilage cells), and myocytes (muscle cells). The ADRCs also secrete growth factors, cytokines, and extracellular matrix proteins that aid in tissue repair and regeneration.

In some embodiments, the ADRCs can be administered by injection or other suitable administration. The ADRCs can be administered in an amount of 1 million to 100 million cells, 5 million to 75 million cells, 10 million to 60 million cells, or 15 million to 50 million cells, or 20 million to 40 million cells, or about 30 million cells.

In some embodiments, the ADRC can be harvested as described herein and propagated. As such, a harvested ADRC can be inoculated into a cell culture and grown in a cell culture environment. The cell culture can include a cell culture medium that is suitable for ADRCs. To grow and propagate ADSCs without inducing differentiation, specific media formulations are used. These media formulations are designed to maintain the cells in their undifferentiated state while promoting their proliferation. Examples of propagation media include DMEM (Dulbecco's Modified Eagle Medium), high glucose DMEM, low glucose DMEM, fetal bovine serum (FBS) at a percentage (e.g., 10-20% FBS), antibiotic (e.g., penicillin, streptomycin, etc.), glutamine, non-essential amino acids, basic fibroblast growth factor (bFGF), and other ingredients. In some aspects, the cells can undergo culture for 1 to 7 days, or other time period from isolation to reimplantation. The cells may undergo passages, such as 1, 2, 3, 4, 5, 6, 7, 8, 9, or 10 passages prior to being reimplanted after isolation.

### Cell Preparation System

In some embodiments, the ADRCs can be separated from adipose or liposuction composition using any sufficient protocol or separation device. The adipose or liposuction compositions can be treated with a digestion composition to digest the adipose tissue. The digested adipose tissue can be washed to remove material from the cells, and stromal stem cells and other progenitor calls can be concentrated. These concentrated cells can be further isolated or used for the treatments described herein.

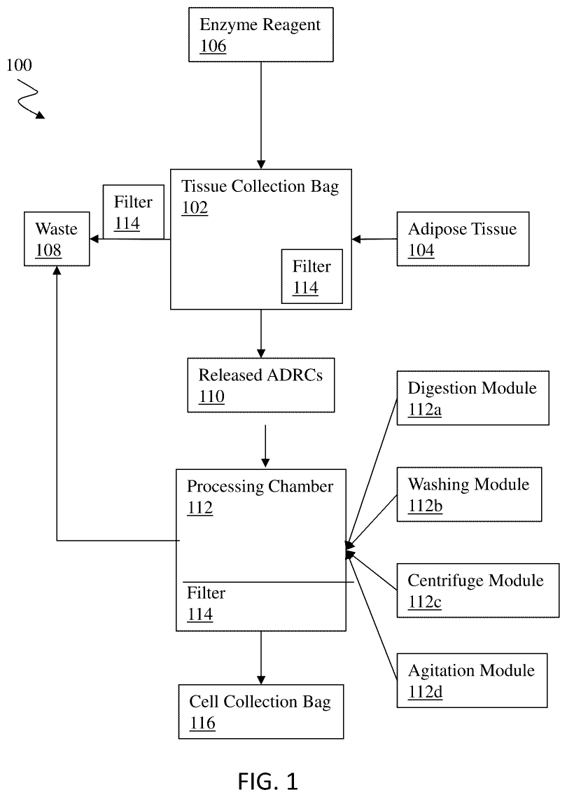

In some embodiments, the cell preparation system can include the Celution® system (e.g., Celution® device, Celution® consumables set, and Celase® reagent, from Cytori or Plus Therapeutics), which can digest adipose tissue in order to further extract, wash and concentrate stromal stem cells and other associated progenitor cells that can be used in a method for autologous reimplantation for treatment of IBM. The Celution® device and system may be referred to as a cell preparation device and system. The cell preparation device is intended to be used in conjunction with the cell preparation consumable set and cell preparation enzyme mixture to digest human adipose tissue in order to perform the extraction. The cell preparation enzyme mixture (e.g., Celase reagent) is intended to digest and liquefy the adipose tissue matrix for the purpose of releasing ADRCs from the adipose tissue matrix during processing with the cell preparation device. As shown herein in FIG. 1, the cell preparation system 100 can be used to safely and efficiently process an autologous graft consisting of adipose-derived regenerative cells (ADRCs) sufficiently to be used in the treatment of IBM as described herein.

FIG. 1 shows the cell preparation system 100 to include a tissue collection bag 102, which can be a sterile bag for collecting and transporting the harvested adipose 104 tissue from the patient. An enzyme reagent 106 can include sterile enzyme solution used to digest the adipose tissue to release the ADRCs 110. The waste digested adipose material can go to the waste 108. A processing chamber 112 can include a sterile, single-use chamber where the released ADRCs and any adipose tissue is processed, including digestion (e.g., digestion module 112a), washing (e.g., washing module 112b), and separation (e.g., centrifuge module 112c and filter 114) steps. Centrifuge tubes (not shown) can include sterile tubes used during the centrifugation steps to separate the ADRCs from the digested tissue solution. A wash solution supplied by the washing module 112b can include any sterile solutions (e.g., aqueous) used to wash the cells during the processing procedure to remove impurities and ensure the purity of the isolated ADRCs. A cell collection bag 116 can include a sterile bag for collecting the concentrated ADRCs after processing, and for providing the ADRCs ready for therapeutic use. Also, sterile filters 114 and connectors can be included to ensure that fluids are properly transferred between different components of the system while maintaining sterility. Transfer tubing (e.g., shown by the arrows) can include sterile tubing used to transfer fluids and cells between different parts of the system during processing. Disposable sterile containers and solutions can be used for resuspending the concentrated ADRCs in a medium suitable for injection or infusion into the patient. All components are provided in sterile packaging to maintain their sterility until use.

The Celution device, developed by Cytori Therapeutics (now known as Plus Therapeutics), is an example of an automated medical device designed for processing adipose (fat) tissue to isolate and concentrate adipose-derived regenerative cells (ADRCs). These cells are then used for various therapeutic applications in regenerative medicine. The device automates the process of isolating ADRCs from adipose tissue, reducing manual handling and the risk of contamination. The device operates within a closed system to maintain sterility and minimize the risk of contamination throughout the cell processing procedure. The device can process adipose tissue and isolate ADRCs in a relatively short time, typically within one to two hours, allowing for same-day therapeutic applications. The device is designed with an intuitive interface that guides clinicians through each step of the processing procedure.

In some embodiments, the cell preparation system can include the following elements. A tissue collection bag can include a sterile bag for collecting and transporting the harvested adipose tissue from the patient. An enzyme reagent can include sterile enzyme solution used to digest the adipose tissue, breaking down the extracellular matrix to release the regenerative cells. A processing chamber can include a sterile, single-use chamber where the adipose tissue is processed, including digestion, washing, and separation steps. Centrifuge tubes can include sterile tubes used during the centrifugation steps to separate the ADRCs from the digested tissue solution. A wash solution can include any sterile solutions (e.g., aqueous) used to wash the cells during the processing procedure to remove impurities and ensure the purity of the isolated ADRCs. A cell collection bag can include a sterile bag for collecting the concentrated ADRCs after processing, ready for therapeutic use. Also, sterile filters and connectors can be included to ensure that fluids are properly transferred between different components of the system while maintaining sterility. Transfer tubing can include sterile tubing used to transfer fluids and cells between different parts of the system during processing. Disposable sterile containers and solutions can be used for resuspending the concentrated ADRCs in a medium suitable for injection or infusion into the patient. All components are provided in sterile packaging to maintain their sterility until use.

The cell preparation system can be an electromechanical medical device including a centrifuge, tissue collection canister agitator, peristaltic pumps, pinch valves, various fault sensors, FPGA (Field Programmable Gate Array) controller and associated firmware logic, user interface keypad and associated vacuum fluorescent display. The cell preparation system can include a device that weighs approximately 99 Kg, measures 96 cm (35″)×60 cm (20″) with a height of 197 cm (55″) and can be equipped with lockable caster wheels for portability in the operating suite.

The cell preparation device can be used in conjunction with a sterile disposable set that includes a lipoaspirate tissue collection container (e.g., 102, 112) with 265 μm filter mesh (e.g., 114), a processing chamber (e.g., 112), a waste bag (e.g., 108), and associated tubing (e.g., arrows). Various washing solutions (typically normal saline or Ringer's lactate) can be provided by the user and connected to the single-use disposable set through the use of “spike ports” attached to the ends of the tubing. The concentration chamber interfaces with the centrifuge through use of a “rotating seal” that consists of a stainless-steel mount that mates with the stainless-steel shaft of the centrifuge.

The agitator of the cell processing device gently mixes the adipose tissue and enzyme reagent to digest the tissue within a collection container. The centrifuge of the cell preparation device concentrates and pellets the cells in the processing chamber, and the peristaltic pumps and associated pinch valves interact with the consumable tubing to remove the supernatant from the pelleted cells and deliver the waste to the waste bag. The cell preparation device then washes the cells by delivering the user-provided saline or Ringer's lactate to the pelleted cells through the use of the same peristaltic pump/tubing interface and centrifugation of the processing chamber. Upon completion of the wash cycles, the pelleted cells are then available for removal by the user through the use of user provided syringe and needle.

The cell preparation device can digest adipose tissue with the enzyme (e.g., Celase) reagent and extract stem cells and progenitor cells. The device subsequently moves cells and fluids through a closed system of tubing and reservoirs, concentrates cells through the use of centrifugation technology, and subsequently removes the waste through the use of the same pumps and tubing used to deliver the fluids and cells. During the cell concentration process, the user may concurrently wash a segregated amount of freshly collected adipose tissue. When cell processing is complete, the user will introduce the concentrated cell pellet from the processing chamber into the washed adipose tissue in the collection container (e.g., cell collection bag). The agitator (e.g., agitation module 112d) will then proceed to gently rotate the cell/tissue suspension to achieve a mixture which will then be available for immediate re-implantation.

The extraction of stem cells and progenitor cells from adipose tissue occurs in the collection container. The agitator of the cell preparation device gently rotates the collection container and mixes the adipose tissue and enzyme reagent to digest the tissues within the collection container. The 265 μm filter mesh within the collection container entraps the adipose tissue introduced into the canister and allows for contaminating red blood cells (RBCs) and washing solutions to pass through the filter mesh and into the waste bag while allowing the washed adipose tissue to remain within the canister on the other side of the filter mesh. This allows for the digestion of the washed adipose tissue and subsequent release of stem cells and associated progenitor cells through use of the Celase reagent which digests the adipose tissue and releases the stem cells and associated progenitor cells entrapped within the adipose matrix. The adipose waste tissue and stem cells and associated progenitor cells are separated based on buoyancy, as the adipose waste floats to the top of the aqueous solutions, while the non-buoyant stem cells and associated progenitor collect at the bottom of the collection container. The stem cells and associated progenitor cells move to the processing chamber where the cellular product is washed and concentrated for eventual removal by the operator through the use of a syringe and needle.

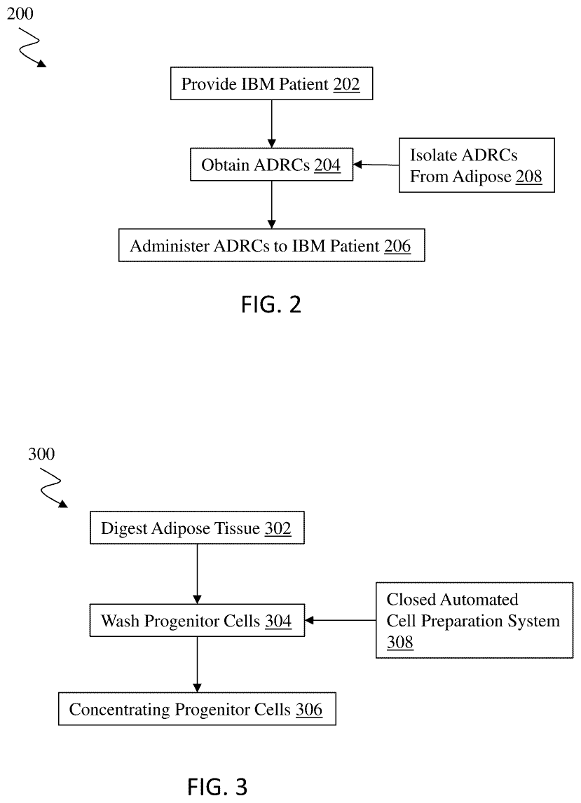

FIG. 2 illustrates a method 200 for treating inclusion body myositis (IBM) in a subject. The method 200 can include providing a subject that has IBM, such as a patient diagnosed with IBM at block 202. The method 200 can include obtaining adipose-derived regenerative cells (ADRC) at block 204. The method 200 can include administering the ADRC to the subject to treat the IBM at block 206. In some aspects, the method 200 can include obtaining the ADRC from the subject prior to administering the ADRC, or the cells may be isolated from a different subject. The different subject may be matched to the IBM patient by having same blood type, is a relative, or other characteristic. In some aspects, the method 200 can include isolating the ADRC from adipose tissue at block 208. In some aspects, the method can include using a closed automated system to isolate the ADRC.

FIG. 3 illustrates a method 300 of obtaining purified ADRCs. The method 300 is performed by a closed automated system that isolates the ADRC by: digesting adipose tissue with a collagenase enzyme to extract progenitor cells at block 302; washing the extracted progenitor cells at block 304; and concentrating the progenitor cells by centrifugation at block 306. In some aspects, the method 300 is performed on a closed automated system 308 that includes a software-driven electro-mechanical centrifuge for separation and concentrating the progenitor cells.

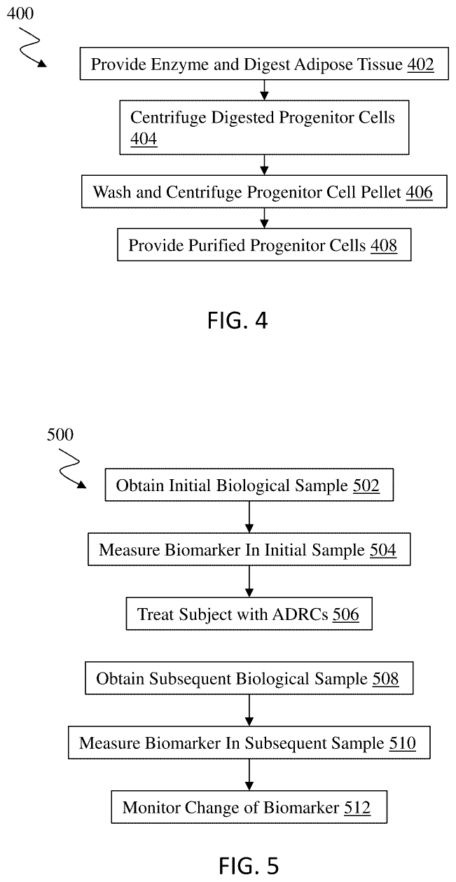

FIG. 4 illustrates a method 400 of isolating ADRCs in an automated closed system. The method 400 can include digesting adipose tissue using a proteolytic enzyme blend to disrupt adipose tissue matrix and release the ADRC at block 402. The method 400 can include centrifuging the digested ADRC to obtain a pellet thereof at block 404. The method 400 can include washing the pellet of ADRC and centrifuging the ADRC through at least one cycle of washing and centrifuging at block 406. The method 400 can include providing the ADRC in purified form at block 408.

In some embodiments, the method of obtaining the ADRCs can include extracting the ADRC from adipose tissue in a collection container and using an agitator that rotates the collection container and mixes the adipose tissue and digestion reagent to digest the adipose tissue within the collection container. Filtering can be performed with a filter mesh within the collection container entraps the adipose tissue, which allows red blood cells (RBCs) and washing solutions to pass through the filter mesh and into a waste bag while allowing the washed adipose tissue to remain within the collection container on the other side of the filter mesh, thereby providing digestion of the washed adipose tissue and subsequent release of the ADRC. Filtering, washing, and/or centrifuging can be used for separating adipose waste tissue from the ADRC based on buoyancy, as the adipose waste tissue floats to the top of an aqueous solution, while the non-buoyant ADRC collect at the bottom of the collection container. The ADRCs can be moved to a processing chamber, and washing and concentrating the ADRC can occur in the processing chamber. The concentrated ADRCs can be removed from the processing chamber.

FIG. 5 illustrates a method 500 of monitoring treatment of IBM with ADRCs. The method 500 can include obtaining at least one initial biological sample from the subject before administering the ADRC at block 502. The method 500 can include measuring the biomarker TDP-43 and/or biomarker p62 in the at least one initial biological sample at block 504. The subject is treated with ADRCs at block 506. The method 500 includes obtaining at least one subsequent biological sample from the subject after administering the ADRC at block 508. The method can include measuring the biomarker TDP-43 and/or biomarker p62 in the at least one subsequent biological sample at block 510. The method can include monitoring a change in the biomarker TDP-4 and/or biomarker P62 from the at least one initial biological sample to the at least one subsequent biological sample as an indication of effectiveness of the treatment at block 512.

### Methods

In some embodiments, the present invention includes the use of adipose-derived regenerative cells (ADRCs) in the treatment of muscle weakness in IBM patients. In some aspects, the treatment includes injection of adipose-derived regenerative cells for IBM treatment. The injection can include autologous reimplantation of ADRCs intramuscularly into the forearm (e.g., flexor digitorum profundus) and/or quadriceps group of muscles for treatment. The treatment can be performed with the injection of the ADRCs in an amount sufficient to slow, stabilize, or even reverse the progression of muscle weakness in patients with IBM.

In some embodiments, the administration of the ADRCs can be into a muscle affected or impacted by the subject having IBM. In some aspects, the injection can be into the quadriceps (e.g., thigh muscles), which are the muscles at the front of the thighs. In some aspects, treatment with ADRCs can improve the quadriceps, which can include an improvement in activities such as walking, climbing stairs, and standing up from a seated position. The ability to perform the activities can be assessed prior to treatment, at the time of treatment, and/or after treatment to monitor any change in ability to perform the activities.

In some embodiments, the injection is into finger flexor muscles in the forearm. Injection into the finger flexor muscles can improve bending of the fingers and gripping objects. Additionally, the treatment can lead to improvements of tasks that require fine motor skills, such as buttoning a shirt or holding a pen.

In some embodiments, the injection is into the hand and wrist muscles. The treatment can lead to improvement in these muscles, which can include improvement in the movement and strength of the hands and wrists, contributing to improved grip strength and manual dexterity.

In some embodiments, the injection can be into the biceps or triceps, which are upper arm muscles. Injection of the ADRCs into the biceps and triceps muscles at the front of the upper arms can improve lifting and carrying objects and performing activities such as pulling or pushing.

In some embodiments, the injection can be into the anterior tibialis, which are the lower leg muscles. The injection can be into the muscles at the front of the lower legs, which can improve the ability of the subject for lifting the foot and toes. Also, improvements can be in ability for walking and balance.

In some embodiments, the injection can be into the neck flexors and extensors muscles. Injection to the neck muscles can improve the subject's ability to control the movement and stability of the neck.

In some embodiments, the injection can be into the throat muscles, such as the pharyngeal muscles. Injection to the pharyngeal muscles can result in improved swallowing. Accordingly, the ADRCs can be used to treat IBM-related dysphagia (e.g., difficulty swallowing).

In some embodiments, the cell separation protocol can include liposuction where adipose tissue is harvested from the patient through a liposuction procedure. Tissue processing can include placing the harvested adipose tissue into the cell preparation device, where it undergoes enzymatic digestion using a proprietary enzyme blend (e.g., Celase) to break down the extracellular matrix and release the cells. Cell isolation can include the device separating and concentrating the ADRCs from the digested tissue through a series of washing and centrifugation steps. Cell concentration occurs from centrifugation, resulting ADRC pellet is resuspended in a solution suitable for injection or infusion. The concentrated ADRCs are ready for immediate therapeutic use in the treatment of IBM.

In some embodiments, the automated and closed system of the device enhances the efficiency and safety of processing ADRCs, providing a quick and reliable method for obtaining regenerative cells. By minimizing manual handling and maintaining a closed environment, the risk of contamination and infection is reduced, which allows for either immediate use or storage (e.g., cryogenic storage). The ability to process and use ADRCs on the same day facilitates timely therapeutic intervention of IBM.

### Muscle Testing:

Muscle testing can be performed bilaterally by different methods for accessing muscle strength at a time point, which can be used over multiple time points to track development or treatment the IBM in the subject. Various testing methods are described herein. However, future-developed testing methods can also be used.

In some embodiments, a muscle test method can include manual muscle testing (MMT). Full and brief MMT can be performed at any stage prior to, during, or after administration of the ADRCs. Full MMT can include bilateral neck flexion/extension, shoulder abduction, elbow flexion/extension, wrist flexion/extension, finger flexion/extension, hip flexion/extension, hip abduction/adduction, knee flexion/extension, and ankle flexion/extension. Brief MMT can include bilateral knee extension, hip flexion, shoulder abduction, and finger flexion on the Kendall modification of the MRC scale being 0-10 scale.

In some embodiments, full MMT can be performed at screening visit prior to ADRC administration, Month 6, Month 12, Month 18, and Month 24. Brief MMT can be performed at baseline prior to ADRC administration, Month 1, Month 3, and Month 9. This method is routinely performed in a clinical setting and has been shown to be reliable. Manual muscle testing will be performed by the PI.

In some embodiments, muscle testing includes using the MicroFET2 hand myometer, which is a hand-held device that allows the practitioner to push against a muscle while the patient resists. This device provides an actual measurement number that can be tracked, such as prior, during, and after ADRC administration. Knee extension can be tested bilaterally. Each movement can be tested twice or more while the patient is encouraged by the practitioner to exert maximal effort.

In some embodiments, a strength test method can use a Martin Vigorimeter that will measure grip strength. As such, the grip strength can be measured and tracked over the course of the treatment.

### IBM Functional Rating Scale (IBMFRS):

In some embodiments, the IBMFRS can be measured. The IBMFRS is a quickly administered (10-minute) ordinal rating scale used to determine a participant's assessment of their capability and independence. It includes 10 measures (e.g., swallowing, handwriting, cutting food and handling utensils, fine motor tasks, dressing, hygiene, turning in bed and adjusting covers, changing position from sitting to standing, walking, and climbing stairs), graded on a Likert scale from 0 (being unable to perform) to 4 (normal). The sum of the 10 items gives a value between 0 and 40, with a higher score representing less functional limitation.

### Modified Timed Up and Go:

In some embodiments, a patient's ability to get up from a chair allowing participants to use their arms can be measured. Most with s-IBM cannot perform the task without pushing off. Also, the participant can walk 3 meters, turn around and walk back to the chair and sit down. The use of nearby walls, or assistance from a caregiver is not allowed.

### Health Assessment Questionnaire-Disability Index:

In some embodiments, the subject will be asked a number of questions about their health in a questionnaire. This is a self-report functional status measure based on the patient-centered dimension of disability. The Health Assessment Questionnaire-Disability Index (HAQ-DI) is a widely used tool designed to assess the degree of disability in patients with rheumatic diseases. It evaluates a patient's ability to perform daily activities and includes questions related to different aspects of physical functioning.

### Patient Global Impression of Change:

In some embodiments, the Patient Global Impression of Change (PGIC) can be performed, which is an assessment by the subject of self-perceived change in ability to conduct daily activities since the start of study medication. The scale ranges from ‘very much worse’ to ‘much worse,’ ‘a little worse,’ ‘no change,’ ‘a little improved,’ ‘much improved,’ and ‘very much improved’.

### EXAMPLES

See: Inclusion Body Myositis Treatment With Celution Processed Adipose Derived Regenerative Cells—Full Text View—ClinicalTrials.gov (clinicaltrials.gov/ct2/show/NCT04975841), which is incorporated herein by specific reference in its entirety. This identifies a clinical study evaluating the efficacy of utilizing the Celution system to isolate adipose-derived regenerative cells for use in treatment of muscle weakness resulting from Inclusion Body Myositis.

See: classic.clinicaltrials.gov/ct2/show/NCT05032131?term=adipose+derived+regenerative+cells&cond=Inclusion+Body+Myositis&draw=2&rank=2. A clinical study evaluating the tolerance and efficacy of treating Inclusion Body Myositis by intramuscular injection of autologous uncultured adipose-derived stromal vascular fraction cells in the non-dominant forearm. This describes a clinical study evaluating the tolerance and efficacy of treating Inclusion Body Myositis by intramuscular injection of autologous uncultured adipose-derived stromal vascular fraction cells in the non-dominant forearm.

The treatment of IBM can be monitored by the biomarker TDP43 and/or biomarker p62. A biological sample can be obtained at any point, including before treatment with ADRCs, at treatment, or subsequent to treatment. The samples can be any type of sample, such as blood, plasma, urine, feces, sweat, tears, or other.

The TDP43 analysis can be based on assumption that phosphorylated TDP43 is at a higher level in IBM patient blood, such as in platelets in the blood. The assay can measure TDP43 phosphorylation, and track the phosphorylation over the treatment period. The biomarker can be measured by any method, such as Simple Western assay.

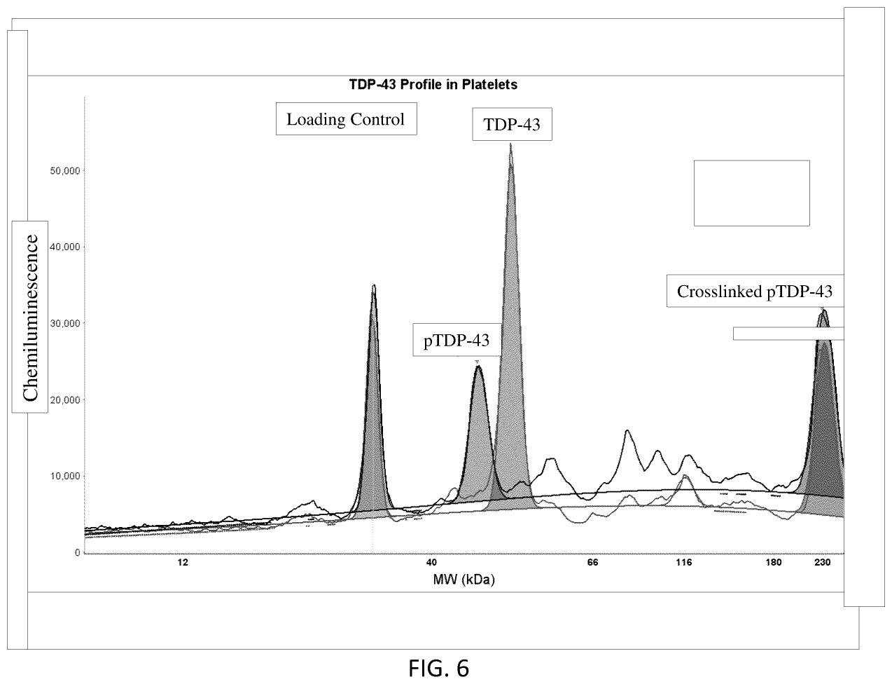

An example of measurement data for the biomarker TDP43 (TDP-43) is shown in FIG. 6. As shown, the size is shown with chemiluminescence for the loading control, pTDP-43, TDP-43, and crosslinked pTDP-43 or crosslinked TDP-43. This allows measuring the amount of TDP43 at each time point so that whether or not the TDP43 is increasing, decreasing, or staying the same. It is expected that treatment with ADRCs decreases phosphorylation of TDP43, where the decrease is an indication that the treatment is working, and the patient can improve.

A similar measurement can be performed with biomarker p62.

### Embodiments

Embodiment 1. A method for treating inclusion body myositis (IBM) in a subject, comprising: providing a subject that has IBM; obtaining adipose-derived regenerative cells (ADRC); and administering the ADRC to the subject to treat the IBM.

Embodiment 2. The method of one of the embodiments, comprising obtaining the ADRC from the subject prior to administering the ADRC.

Embodiment 3. The method of one of the embodiments, further comprising isolating the ADRC from adipose tissue.

Embodiment 4. The method of one of the embodiments, further comping using a closed automated system to isolate the ADRC.

Embodiment 5. The method of Embodiment 4, wherein the closed automated system isolates the ADRC by: digesting adipose tissue with a collagenase enzyme to extract progenitor cells; washing the extracted progenitor cells; and concentrating the progenitor cells by centrifugation.

Embodiment 6. The method of Embodiment 4, wherein the closed automated system includes a software-driven electro-mechanical centrifuge for separation and concentrating the progenitor cells.

Embodiment 7. The method of one of the embodiments, comprising: injecting the ADRC as cells into a plurality of injection locations to provide at least a million cells, more preferably at least 10 million cells, even more preferably at least 20 million cells, or most preferably at least 30 million cells.

Embodiment 8. The method of one of the embodiments, comprising: performing a safety assessment of the subject after the injection, which safety assessment includes observations of adverse events, vital signs, and lab values of the subject.

Embodiment 9. The method of Embodiment 8, comprising monitoring the treatment with the ADRC by one or more of: IBM Functional Rating Scale (IBMFRS); Modified Timed Up and Go (mTUG); Manual Muscle Testing (MMT); Grip Strength Martin Vigorimeter; Quadricep strength-HHD; or Health Assessment Questionnaire-Disability Index (HAQ-DI).

Embodiment 10. The method of one of the embodiments, further comprising monitoring the treatment by monitoring a biomarker before and after the administering of the ADRC, wherein the biomarker includes TDP-43.

Embodiment 11. The method of Embodiment 10, comprising: obtaining at least one initial biological sample from the subject before administering the ADRC; measuring the biomarker TDP-43 in the at least one initial biological sample; obtaining at least one subsequent biological sample from the subject after administering the ADRC; measuring the biomarker TDP-43 in the at least one subsequent biological sample; and monitoring a change in the biomarker TDP-4 from the at least one initial biological sample to the at least one subsequent biological sample as an indication of effectiveness of the treatment.

Embodiment 12. The method of one of the embodiments, wherein the administering includes injecting cells into a muscle of the subject.

Embodiment 13. The method of Embodiment 12, wherein the muscle is selected from quadriceps muscles, finger flexor muscles, hand muscles, wrist muscles, biceps muscles, triceps muscles, anterior tibialis muscles, neck flexor muscles, neck extensor muscles, pharyngeal muscles, and combinations thereof.

Embodiment 14. The method one of the embodiments, wherein the administration is into a joint that is associated with pain in the subject.

Embodiment 15. The method of Embodiment 14, wherein the administration is into a knee joint, elbow joint, finger joint, wrist joint, ankle joint, shoulder joint, or combinations thereof.

Embodiment 16. The method of one of the embodiments, comprising in an automated closed system: digesting adipose tissue using a proteolytic enzyme blend to disrupt adipose tissue matrix and release the ADRC; centrifuging the digested ADRC to obtain a pellet thereof; washing the pellet of ADRC and centrifuging the ADRC through at least one cycle of washing and centrifuging; and providing the ADRC in purified form.

Embodiment 17. The method of Embodiment 10, comprising monitoring a TDP-43 accumulation in one or more muscle locations in the subject.

Embodiment 18. The method of Embodiment 17, comprising monitoring TDP-43 in myofibers of the subject that are associated with IBM.

Embodiment 19. The method of one of the embodiments, comprising: obtaining at least one initial biological sample from the subject before administering the ADRC; measuring the biomarker p62 in the at least one initial biological sample; obtaining at least one subsequent biological sample from the subject after administering the ADRC; measuring the biomarker p62 in the at least one subsequent biological sample; and monitoring a change in the biomarker p62 from the at least one initial biological sample to the at least one subsequent biological sample as an indication of effectiveness of the treatment.

Embodiment 20. The method of one of the embodiments, comprising: extracting the ADRC from adipose tissue in a collection container; using an agitator that rotates the collection container and mixes the adipose tissue and digestion reagent to digest the adipose tissue within the collection container; filtering with a filter mesh within the collection container entraps the adipose tissue, which allows red blood cells (RBCs) and washing solutions to pass through the filter mesh and into a waste bag while allowing the washed adipose tissue to remain within the collection container on the other side of the filter mesh, thereby providing digestion of the washed adipose tissue and subsequent release of the ADRC; separating adipose waste tissue from the ADRC based on buoyancy, as the adipose waste tissue floats to the top of an aqueous solution, while the non-buoyant ADRC collect at the bottom of the collection container; moving the ADRC to a processing chamber; washing and concentrating the ADRC in the processing chamber; and removing the concentrated ADRC from the processing chamber.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

As used herein, the term “treat” or “treated” or “treating” or “treatment” refer to any type of action that imparts a modulating effect, which, for example, can be a beneficial effect, to a subject afflicted with a disorder, disease, or condition (e.g., BMI or symptoms), including improvement in the condition of the subject (e.g., in one or more symptoms), delay or reduction in the progression of the condition, and/or change in clinical parameters, disease or illness, etc., as would be well known in the art. The term “treatment” includes therapeutic and/or prophylactic treatment of IBM, the diminishment or alleviation of at least one symptom associated with IBM, and the eradication of one or more symptoms of IBM.

In view of the foregoing, the present technology provides pharmaceutical compositions and medicaments comprising any one or more of the compounds of the adjuvants, or derivative thereof, prodrug thereof, as disclosed herein and optionally a pharmaceutically acceptable carrier or one or more excipients or fillers. The compositions may be used in the methods and treatments described herein. Such compositions and medicaments include a therapeutically effective amount of any compound as described herein to function as a therapy for IBM. The pharmaceutical composition may be packaged in unit dosage form.

The pharmaceutical compositions and medicaments may be prepared by mixing one or more compounds of the present technology with pharmaceutically acceptable carriers, excipients, binders, diluents or the like. The compounds and compositions described herein may be used to prepare formulations and medicaments to treat IBM. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology. The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the present technology.

Those skilled in the art are readily able to determine an effective amount, such as by simply administering a compound of the present technology to a patient in increasing amounts of the adjuvant until the identification of a vaccine composition with the proper amount of adjuvant.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

### REFERENCES

1. Amato A A and Barohn R J. Evaluation and treatment of inflammatory myopathies. J Neurol Neurosurg Psychiatry. 2009; 80:1060-8.
2. Amato A A and Barohn R J. Inclusion body myositis: old and new concepts. J Neurol Neurosurg Psychiatry. 2009; 80:1186-93.
3. Amato A A, Gronseth G S, Jackson C E, Wolfe G I, Katz J S, Bryan W W and Barohn R J. Inclusion body myositis: clinical and pathological boundaries. Ann Neurol. 1996; 40:581-6.
4. Dimachkie M M and Barohn R J. Inclusion body myositis. Neurol Clin. 2014; 32:629-46, vii.
5. Engel W K and Askanas V. Inclusion-body myositis: clinical, diagnostic, and pathologic aspects. Neurology. 2006; 66: S20-9.
6. Griggs R C, Askanas V, DiMauro S, Engel A, Karpati G, Mendell J R and Rowland L P. Inclusion body myositis and myopathies. Ann Neurol. 1995; 38:705-13.
7. Machado P M, Ahmed M, Brady S, Gang Q, Healy E, Morrow J M, Wallace A C, Dewar L, Ramdharry G, Parton M, Holton J L, Houlden H, Greensmith L and Hanna M G. Ongoing developments in sporadic inclusion body myositis. Curr Rheumatol Rep. 2014; 16:477.
8. Machado P M, Dimachkie M M and Barohn R J. Sporadic inclusion body myositis: new insights and potential therapy. Curr Opin Neurol. 2014; 27:591-8.
9. Tawil R and Griggs R C. Inclusion body myositis. Curr Opin Rheumatol. 2002; 14:653-7.
10. Amato A A and Barohn R J. Idiopathic inflammatory myopathies. Neurol Clin. 1997; 15:615-48.
11. Dimachkie M M. Idiopathic inflammatory myopathies. J Neuroimmunol. 2011; 231:32-42.
12. Dimachkie M M and Barohn R J. Inclusion body myositis. Curr Neurol Neurosci Rep. 2013; 13:321.
13. Dimachkie M M and Barohn R J. Inclusion body myositis. Semin Neurol. 2012; 32:237-45.
14. Dimachkie M M and Barohn R J. Idiopathic inflammatory myopathies. Semin Neurol. 2012; 32:227-36.
15. Mastaglia F L, Garlepp M J, Phillips B A and Zilko P J. Inflammatory myopathies: clinical, diagnostic and therapeutic aspects. Muscle Nerve. 2003; 27:407-25.
16. Phillips B A, Cala L A, Thickbroom G W, Melsom A, Zilko P J and Mastaglia F L. Patterns of muscle involvement in inclusion body myositis: clinical and magnetic resonance imaging study. Muscle Nerve. 2001; 24:1526-34.
17. Dimachkie M M and Barohn R J. Idiopathic inflammatory myopathies. Front Neurol Neurosci. 2009; 26:126-146.
18. Benveniste O, Guiguet M, Freebody J, Dubourg O, Squier W, Maisonobe T, Stojkovic T, Leite M I, Allenbach Y, Herson S, Brady S, Eymard B and Hilton-Jones D. Long-term observational study of sporadic inclusion body myositis. Brain. 2011; 134:3176-84.
19. Griggs R C. The current status of treatment for inclusion-body myositis. Neurology. 2006; 66: S30-2.
20. Rose M R, McDermott M P, Thornton C A, Palenski C, Martens W B and Griggs R C. A prospective natural history study of inclusion body myositis: implications for clinical trials. Neurology. 2001; 57:548-50.
21. Cox F M, Titulaer M J, Sont J K, Wintzen A R, Verschuuren J J and Badrising U A. A 12-year follow-up in sporadic inclusion body myositis: an end stage with major disabilities. Brain. 2011; 134:3167-75.
22. Price M A, Barghout V, Benveniste O, Christopher-Stine L, Corbett A, de Visser M, Hilton-Jones D, Kissel J T, Lloyd T E, Lundberg I E, Mastaglia F, Mozaffar T, Needham M, Schmidt J, Sivakumar K, DeMuro C and Tseng B S. Mortality and Causes of Death in Patients with Sporadic Inclusion Body Myositis: Survey Study Based on the Clinical Experience of Specialists in Australia, Europe and the USA. J Neuromuscul Dis. 2016; 3:67-75.
23. Lilleker J B, Rietveld A, Pye S R, Mariampillai K, Benveniste O, Peeters M T, Miller J A, Hanna M G, Machado P M, Parton M J, Gheorghe K R, Badrising U A, Lundberg I E, Sacconi S, Herbert M K, McHugh N J, Lecky B R, Brierley C, Hilton-Jones D, Lamb J A, Roberts M E, Cooper R G, Saris C G, Pruijn G J, Chinoy H, van Engelen B G and all Uc. Cytosolic 5′-nucleotidase 1A autoantibody profile and clinical characteristics in inclusion body myositis. Ann Rheum Dis. 2017; 76:862-868.
24. Estephan B B R, Dimachkie M M. Sporadic IBM: a Case Cohort. Journal of Clinical Neuromuscular Disease. 2011; 12:18-19.
25. Salajegheh M, Pinkus J L, Taylor J P, Amato A A, Nazareno R, Baloh R H and Greenberg S A. Sarcoplasmic redistribution of nuclear TDP-43 in inclusion body myositis. Muscle Nerve. 2009; 40:19-31.
26. Subourg O W J, Maisonobe T. Diagnositic value of markers of muscule degeneration in sporadic inclusion body myositis. Acta Myol. 2011; 30:103-108.
27. Askanas V and Engel W K. Molecular pathology and pathogenesis of inclusion-body myositis. Microsc Res Tech. 2005; 67:114-20.
28. Askanas V, Engel W K and Nogalska A. Sporadic inclusion-body myositis: A degenerative muscle disease associated with aging, impaired muscle protein homeostasis and abnormal mitophagy. Biochim Biophys Acta. 2015; 1852:633-43.
29. Askanas V and Engel W K. Inclusion-body myositis: a myodegenerative conformational disorder associated with Abeta, protein misfolding, and proteasome inhibition. Neurology. 2006; 66: S39-48.
30. Fratta P, Engel W K, McFerrin J, Davies K J, Lin S W and Askanas V. Proteasome inhibition and aggresome formation in sporadic inclusion-body myositis and in amyloid-beta precursor protein-overexpressing cultured human muscle fibers. Am J Pathol. 2005; 167:517-26.
31. Ozturk A M J, Engel W K, Askanas V. HSP70 chaperone machinery in sporadic inclusion-body myositis muscle fibers. Neurology. 2004; 62:168.
32. Joo H J, Kim J H and Hong S J. Adipose Tissue-Derived Stem Cells for Myocardial Regeneration. Korean Circ J. 2017; 47:151-159.
33. Jorgensen A N, Aagaard P, Frandsen U, Boyle E and Diederichsen L P. Blood-flow restricted resistance training in patients with sporadic inclusion body myositis: a randomized controlled trial. Scand J Rheumatol. 2018; 47:400-409.
34. Karaaltin M V and Baghaki S. Adipose Derived Regenerative Cell Therapy for Treating a Diabetic Wound: A Case Report. Wounds. 2012; 24: e1-5.
35. Perez-Cano R, Vranckx J J, Lasso J M, Calabrese C, Merck B, Milstein A M, Sassoon E, Delay E and Weiler-Mithoff E M. Prospective trial of adipose-derived regenerative cell (ADRC)-enriched fat grafting for partial mastectomy defects: the RESTORE-2 trial. Eur J Surg Oncol. 2012; 38:382-9.
36. Henry T D, Pepine C J, Lambert C R, Traverse J H, Schatz R, Costa M, Povsic T J, David Anderson R, Willerson J T, Kesten S and Perin E C. The Athena trials: Autologous adipose-derived regenerative cells for refractory chronic myocardial ischemia with left ventricular dysfunction. Catheter Cardiovasc Interv. 2017; 89:169-177.

1. A method for treating inclusion body myositis (IBM) in a subject, comprising:
providing a subject that has IBM;
obtaining adipose-derived regenerative cells (ADRC); and
administering the ADRC to the subject to treat the IBM.

2. The method of claim 1, comprising obtaining the ADRC from the subject prior to administering the ADRC.

3. The method of claim 1, further comprising isolating the ADRC from adipose tissue.

4. The method of claim 1, further comping using a closed automated system to isolate the ADRC.

5. The method of claim 4, wherein the closed automated system isolates the ADRC by:
digesting adipose tissue with a collagenase enzyme to extract progenitor cells;
washing the extracted progenitor cells; and
concentrating the progenitor cells by centrifugation.

6. The method of claim 4, wherein the closed automated system includes a software-driven electro-mechanical centrifuge for separation and concentrating the progenitor cells.

7. The method of claim 1, further comprising:
injecting the ADRC as cells into a plurality of injection locations to provide at least a million cells, more preferably at least 10 million cells, even more preferably at least 20 million cells, or most preferably at least 30 million cells.

8. The method of claim 1, further comprising:
performing a safety assessment of the subject after the injection, which safety assessment includes observations of adverse events, vital signs, and lab values of the subject.

9. The method of claim 8, further comprising monitoring treatment with the ADRC by one or more of:
IBM Functional Rating Scale (IBMFRS);
Modified Timed Up and Go (mTUG);
Manual Muscle Testing (MMT);
Grip Strength Martin Vigorimeter;
Quadricep strength-HHD; or
Health Assessment Questionnaire-Disability Index (HAQ-DI).

10. The method of claim 1, further comprising monitoring treatment by monitoring a biomarker before and after the administering of the ADRC, wherein the biomarker includes TDP-43.

11. The method of claim 10, further comprising:
obtaining at least one initial biological sample from the subject before administering the ADRC;
measuring biomarker TDP-43 in the at least one initial biological sample;
obtaining at least one subsequent biological sample from the subject after administering the ADRC;
measuring the biomarker TDP-43 in the at least one subsequent biological sample; and
monitoring a change in biomarker TDP-4 from the at least one initial biological sample to the at least one subsequent biological sample as an indication of effectiveness of the treatment.

12. The method of claim 1, wherein the administering includes injecting cells into a muscle of the subject.

13. The method of claim 12, wherein the muscle is selected from quadriceps muscles, finger flexor muscles, hand muscles, wrist muscles, biceps muscles, triceps muscles, anterior tibialis muscles, neck flexor muscles, neck extensor muscles, pharyngeal muscles, and combinations thereof.

14. The method claim 1, wherein the administration is into a joint that is associated with pain in the subject.

15. The method of claim 14, wherein the administration is into a knee joint, elbow joint, finger joint, wrist joint, ankle joint, shoulder joint, or combinations thereof.

16. The method of claim 1, comprising in an automated closed system:
digesting adipose tissue using a proteolytic enzyme blend to disrupt adipose tissue matrix and release the ADRC;
centrifuging the digested ADRC to obtain a pellet thereof;
washing the pellet of ADRC and centrifuging the ADRC through at least one cycle of washing and centrifuging; and
providing the ADRC in purified form.

17. The method of claim 10, comprising monitoring a TDP-43 accumulation in one or more muscle locations in the subject.

18. The method of claim 17, comprising monitoring TDP-43 in myofibers of the subject that are associated with IBM.

19. The method of claim 1, comprising:
obtaining at least one initial biological sample from the subject before administering the ADRC;
measuring biomarker p62 in the at least one initial biological sample;
obtaining at least one subsequent biological sample from the subject after administering the ADRC;
measuring the biomarker p62 in the at least one subsequent biological sample; and
monitoring a change in the biomarker p62 from the at least one initial biological sample to the at least one subsequent biological sample as an indication of effectiveness of treatment.

20. The method of claim 1, comprising:
extracting the ADRC from adipose tissue in a collection container;
using an agitator that rotates the collection container and mixes the adipose tissue and digestion reagent to digest the adipose tissue within the collection container;
filtering with a filter mesh within the collection container entraps the adipose tissue, which allows red blood cells (RBCs) and washing solutions to pass through the filter mesh and into a waste bag while allowing the washed adipose tissue to remain within the collection container on the other side of the filter mesh, thereby providing digestion of the washed adipose tissue and subsequent release of the ADRC;
separating adipose waste tissue from the ADRC based on buoyancy, as the adipose waste tissue floats to top of an aqueous solution, while non-buoyant ADRC collect at bottom of the collection container;
moving the ADRC to a processing chamber;
washing and concentrating the ADRC in the processing chamber; and
removing the concentrated ADRC from the processing chamber.
